(19) United States
(12) Patent Application Publication
(10) Pub. No.: US 2003/0040509 A1
(43) Pub. Date: Feb. 27, 2003

(54) Methods and compositions for treating diseases associated with excesses in ACE

(75) Inventor: David W. Moskowitz, St. Louis, MO (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: GenoMed, LLC

(21) Appl. No.: 10/213,330

(22) Filed: Aug. 6, 2002

Related U.S. Application Data
(60) Provisional application No. 60/310,064, filed on Aug. 6, 2001.
(60) Provisional application No. 60/347,905, filed on Jan. 15, 2002.
(60) Provisional application No. 60/347,013, filed on Jan. 11, 2002.
(60) Provisional application No. 60/350,563, filed on Jan. 24, 2002.
(60) Provisional application No. 60/352,484, filed on Jan. 30, 2002.
(60) Provisional application No. 60/352,072, filed on Jan. 28, 2002.
(60) Provisional application No. 60/352,074, filed on Jan. 28, 2002.
(60) Provisional application No. 60/378,467, filed on May 8, 2002.
(60) Provisional application No. 60/379,796, filed on May 13, 2002.
(60) Provisional application No. 60/380,741, filed on May 16, 2002.

(51) Int. Cl. A61K 31/56; A61K 31/47

(52) U.S. Cl. 514/171; 514/307

(57) ABSTRACT

Over 40 common diseases, in addition to congestive heart failure (CHF) due to hypertension (HTN) or non-insulin dependent diabetes mellitus (type II diabetes mellitus) (NIDDM), atherosclerotic peripheral vascular disease (ASPVD) due to HTN or NIDDM, and chronic obstructive pulmonary disease; emphysema (COPD), are associated with the ACE D/D genotype and should also respond to an adequate tissue-inhibitory dose of ACE inhibitors such as quinapril. Several of these diseases have now been successfully treated using higher than normal dosages of ACE inhibitors, especially hydrophobic ACE inhibitors, with good outcomes. ACE inhibitors have also been found to be useful in inhibiting apoptosis and aging in general. Dosages that have been utilized are typically greater than quinapril at a dose of 40 to 80 mg/day, i.e. up to 1 mg/kg per day for a “typical” 80 kg patient. New formulations of ACE inhibitors have been developed for these higher dosages, including 80 mg tablets, controlled and/or sustained release formulations, and formulations containing a second active agent such as a diuretic, or a compound such as furosemide 20 mg/day (for creatinine <2.5 mg/dl) or furosemide 40 mg/day (for creatinine >2.5 mg/dl), to prevent fluid retention and congestive heart failure in patients with renal failure. The ACE inhibitors can also be combined with an angiotensin receptor blocker.

This application claims priority to U.S. S. No. 60/310,064 filed Aug. 6, 2001; U.S. S. No. 60/347,905 filed Jan. 15, 2002; U.S. S. No. 60/347,013 filed Jan. 11, 2002; U.S. S. No. 60/350,563 filed Jan. 24, 2002; U.S. S. No. 60/352,484 filed Jan. 30, 2002; U.S. S. No. 60/352,072 filed Jan. 28, 2002; and U.S. S. No. 60/352,074 filed Jan. 28, 2002; U.S. S. No. 60/378,467 filed May 8, 2002; U.S. S. No. 60/379,796 filed May 13, 2002; and U.S. S. No. 60/380,741 filed May 16, 2002.

### BACKGROUND OF THE INVENTION

The present invention is generally in the field of methods and compositions for treatment of chronic disease.

Angiotensin converting enzyme (encoded by the gene DCP1, also known as ACE) catalyses the conversion of angiotensin I to the physiologically active peptide angiotensin II, which controls fluid-electrolyte balance and systemic blood pressure. Because of its key function in the renin-angiotensin system, many association studies have been performed with DCP1. Nearly all studies have associated the presence (insertion, I) or absence (deletion, D) of a 287-bp Alu repeat element in intron 16 with the levels of circulating enzyme or cardiovascular pathophysiologies. Many epidemiological studies suggest that the DCP1*D allele confers increased susceptibility to cardiovascular disease; however, other reports have found no such association or even a beneficial effect. Rieder, et al., Nat Genet 22(1):59-62 (1999), reports the complete genomic sequence of DCP1 from 11 individuals, representing the longest contiguous scan (24 kb) for sequence variation in human DNA, and identifies 78 varying sites in 22 chromosomes that resolved into 13 distinct haplotypes. Of the variant sites, 17 were in absolute linkage disequilibrium with the commonly typed Alu insertion/deletion polymorphism, producing two distinct and distantly related clades.

The insertion/deletion polymorphism in intron 16 of the angiotensin I-converting enzyme (ACE) was first described in 1988 by a French group, and extensively studied since then. It is difficult to say exactly which of the seventeen reported polymorphisms in this region is functional. In any event, the deletion/deletion (D/D) genotype has been associated with a two-fold higher level of ACE activity than the insertion/insertion (I/I) genotype on white blood cell plasma membranes. The insertion/deletion (I/D) heterozygote has an intermediate level of ACE activity. The simplest explanation is that the Alu insertion delays the rate of transcription of the ACE gene. There is evidence that the Alu sequence of 287 base pairs can create a cruciform secondary structure in DNA which can bind nuclear proteins involved in DNA recombination, for example. The Alu sequence may bind RNA polymerase II, retarding its progression along the DNA template. The net effect would be a decrease in messenger RNA levels for ACE. This may well translate into decreased protein levels of the enzyme, and hence decreased overall ACE activity in people with the I/I or I/ID genotype relative to people without the Alu insertion at all, i.e. with the D/D genotype.

An ACE inhibitor blocks the body's angiotensin-converting enzymes (ACE), a protein needed by the body to make angiotensin II which increases blood pressure by narrowing arteries. This process leaves blood vessels more relaxed, which decreases blood pressure and increases the flow of blood and oxygen to the heart. ACE inhibitors improve survival in heart failure when added to conventional treatment. The greatest benefit is seen in those patients with the most severe heart failure. A smaller benefit is seen in patients with mild-to-moderate heart failure. However, despite the improved survival, the prognosis of moderate-to-severe heart failure remains poor. Nevertheless, largely because of the potential survival benefit, most cardiologists now believe that an ACE inhibitor should be added to diuretic therapy in all patients with overt heart failure, even if the heart failure is only mild. Some physicians prescribe ACE inhibitors before diuretics, although there is no trial-based evidence for this approach.

The benefits of treatment are not restricted to survival. The addition of an ACE inhibitor to diuretic therapy improves the control of heart failure, an important symptomatic benefit. This reduces the need for hospitalization and probably improves the patient's quality of life. There may also be economic benefits for the health care system. Since their introduction in the mid-1980s, angiotensin converting enzyme (ACE) inhibitors have become well established for the treatment of hypertension and heart failure. In addition, they slow progression of renal impairment in diabetic and cyclosporine A-induced nephropathy (Padi and Chopra, Pharmacol Res 2002 May;45(5):413-20).

Selection of the patients to be treated is not based on the presence or absence of altered ACE levels or the presence of any of the polymorphisms in the gene, however, but solely on the observation of symptoms in which the known vasodilator properties of the ACE inhibitors have been proven to be useful. These patients are typically treated with relatively low doses of the ACE inhibitors in an amount effective to decrease blood pressure.

It is an object of the present invention to provide a method of selecting patients who would benefit from treatment with ACE inhibitors.

It is another object of the present invention to provide a method of treating patients with chronic disease more effectively.

It is still another object of the present invention to provide new formulations of ACE inhibitors for sustained or controlled release or treatment with high dosages.

### SUMMARY OF THE INVENTION

At least 40 common diseases (see Table 1, odds ratio of 1.0 or greater), in addition to congestive heart failure (CHF) due to hypertension (HTN) or non-insulin dependent diabetes mellitus (type II diabetes mellitus) (NIDDM), atherosclerotic peripheral vascular disease (ASPVD) due to HTN or NIDDM, and chronic obstructive pulmonary disease [emphysema (COPD)], are associated with the ACE D/D genotype and should also respond to an adequate tissue-inhibitory dose of ACE inhibitors such as quinapril. Several of these diseases have now been successfully treated using higher than normal dosages of ACE inhibitors, especially hydrophobic ACE inhibitors, with good outcomes. ACE inhibitors have also been found to be useful in inhibiting apoptosis and aging in general. Dosages that have been utilized are typically greater than the conventional maximal dose of quinapril at a dose of 40 to 80 mg/day, i.e. up to 1 mg/kg per day for a “typical” 80 kg patient. As described herein, the recommended dosage is to administer greater than 80 mg/day of an ACE inhibitor such as quinapril. For example, for treatment of a 80 kg patient with renal failure, the patient is initially treated with quinapril, at 20 mg once a day (at bed-time) at the first clinic visit, to 20 mg twice a day at the second visit (1-2 months later), to 40 mg twice a day (1-2 months later), to 80 mg twice a day (1-2 months later), and then maintained at this dosage. The target dose for maximally effective disease prevention should be a ramipril dose of 0.5 mg/kg/day, (or quinapril 2 mg/kg/day), ie., an amount of an ACE inhibitor effective to inhibit i.e. at inhibiting tissue ACE by greater than 95%.

New formulations of ACE inhibitors have been developed for these higher dosages, including 80 mg tablets, controlled and/or sustained release formulations, and formulations containing a second active agent such as a diuretic, or a compound such as furosemide 20 mg/day (for creatinine <2.5 mg/dl) or furosemide 40 mg/day (for creatinine >2.5 mg/dl), to prevent fluid retention and congestive heart failure in patients with renal failure. The ACE inhibitors can also be combined with an angiotensin receptor blocker to treat hyperkalemia or more completely block the action of angiotensin II in tissues; or with hydrocortisone acetate to prevent hyperkalemia.

Veterinary applications are also described, as well as formulations of ACE inhibitors in animal feed.

### DETAILED DESCRIPTION OF THE INVENTION

### I. Disease Classes to be Treated and Methods of Treatment

A. Selection of Diseases and Disorders to be Treated with ACE Inhibitors.

The association of the ACE D/D with atherosclerotic heart disease was described by Cambien, et al. Nature (1992). It has now been determined that the ACE D/D genotype is associated with a large number of diseases, suggesting that an excess of ACE activity contributes to disease causation and/or progression, as well as with apoptosis and aging in general.

TABLE 1
ACE D/D odds ratios for common diseases.

| | Ethnic/ | | Ethnic/ | |
|---|---|---|---|---|
| | racial | Odds | racial | Odds |
| Disease | group | Ratio | group | Ratio |
| MUSCULAR AND RENAL | | | | |
| DISEASES | | | | |
| Hypertension | 1 | 1.27 | 2 | 1.20 |
| ESRD/HTN | 1 | 1.09 | 2 | 1.33 |
| IDDM | 1 | 1.14 | 2 | 1.13 |
| ESRD/NIDDM | 1 | 1.22 | 2 | 1.41 |
| ESRD/FSGS | 1 | 2.84 | 2 | 0.97 |
| MI | 1 | 1.28 | 2 | 1.10 |
| AAA | 1 | 0.80 | 2 | 1.37 |
| Atrial fibrillation | 1 | 1.02 | 2 | 1.15 |
| Cardiomyopathy | 1 | 1.07 | 2 | 1.25 |
| ASPVD | 1 | 1.03 | 2 | 1.14 |
| CHF | 1 | 1.42 | 2 | 1.15 |
| LVH | 1 | 1.04 | 2 | 1.10 |
| DVT | 1 | 1.22 | 2 | 1.58 |
| METABOLIC DISEASES | | | | |
| Obesity (BMI > 30) | 1 | 1.15 | 2 | 1.08 |
| Gout | 1 | 0.91 | 2 | 1.33 |
| Cholesterol > 200 | 1 | 1.11 | 2 | 1.08 |
| NIDDM/retinopathy | 1 | 1.19 | 2 | 1.05 |
| NIDDM/neuropathy | 1 | 1.11 | 2 | 1.14 |
| PULMONARY DISEASES | | | | |
| Cigarette abuse | 1 | 1.13 | 2 | 1.26 |
| COPD | 1 | 1.19 | 2 | 1.18 |
| Asthma | 1 | 1.16 | 2 | 0.60 |
| GI DISEASES | | | | |
| Peptic ulcer | 1 | 1.00 | 2 | 1.00 |
| Gall stones | 1 | 0.60 | 2 | 1.14 |
| Alcoholic cirrhosis | 1 | 1.20 | 2 | 0.86 |
| Viral hepatitis | 1 | 0.90 | 2 | 0.95 |
| GERD | 1 | 1.14 | 2 | 1.39 |
| Diverticulitis | 1 | 0.97 | 2 | 0.61 |
| Inguinal hernia | 1 | 0.63 | 2 | 1.90 |
| MUSCULO-SKELETAL | | | | |
| DISEASE | | | | |
| Osteoarthritis (DJD) | 1 | 1.25 | 2 | 1.07 |
| RHEUMATOLOGIC DISEASE | | | | |
| Rheumatoid arthritis | 1 | 1.82 | 2 | 1.04 |
| SOLID TUMORS | | | | |
| BPH | 1 | 1.25 | 2 | 1.24 |
| Prostate cancer | 1 | 1.35 | 2 | 1.13 |
| Colon polyps | 1 | 0.80 | 2 | 1.42 |
| Colon cancer | 1 | 1.19 | 2 | 1.22 |
| Lung cancer | 1 | 2.02 | 2 | 1.31 |
| Kidney cancer | 1 | 0.76 | 2 | 2.91 |
| NEUROPSYCHIATRIC DISEASES | | | | |
| Alcohol abuse | 1 | 1.08 | 2 | 0.97 |
| Drug abuse (i.v., i.h.) | 1 | 1.04 | 2 | 1.02 |
| Stroke (CVA) | 1 | 1.17 | 2 | 1.08 |
| TIA/s/p CEA | 1 | 1.18 | 2 | 1.20 |
| Seizures | 1 | 1.27 | 2 | 1.15 |
| Alzheimer's | 1 | 1.89 | 2 | 0.39 |
| Multi-infarct | 1 | 0.81 | 2 | 1.70 |
| dementia | | | | |
| Dementia (NOS) | 1 | 1.62 | 2 | 0.82 |
| Schizophrenia | 1 | 0.93 | 2 | 1.18 |
| Depression | 1 | 1.01 | 2 | 1.14 |
| Bipolar affective | 1 | 3.78 | 2 | 2.33 |
| disorder | | | | |
| OPHTHALMOLOGIC DISEASE | | | | |
| Glaucoma | 1 | 1.32 | 2 | 1.57 |
| SICKLE CELL ANEMIA | | | | |
| Hgb SS | 1 | 1.21 | | |
| Hgb AS | 1 | 1.26 | | |

The gender indicated as x, female; y, male. Race is indicated as 1, African American; 2, American Caucasian; 3, Hispanci.

The following abbreviations are used herein:

| | |
|---|---|
| ESRD | end-stage renal disease |
| HTN | hypertension |
| NIDDM | non-insulin dependent diabetes mellitus (type II diabetes |
| | mellitus) |
| FSGS | focal segmental glomerulosclerosis |
| MI | myocardial infarction |
| ASCAD | atherosclerotic coronary artery disease |
| AAA | abdominal aortic aneurysm |
| ASPVD | atherosclerotic peripheral vascular disease |
| CHF | congestive heart failure |
| LVH | left ventricular hypertrophy |
| DVT | deep vein thrombosis |
| BMI | body mass index |
| Chol > 200 | cholesterol >200 mg/dl |
| COPD | chronic obstructive pulmonary disease; emphysema |
| GI | gastro-enterological |
| GERD | gastro-esophageal reflux disease |
| DJD | degenerative joint disease |
| BPH | benign prostatic hypertrophy/hyperplasia |
| I.v., i.h. | Intravenous; inhalational (drug abuse): e.g. Heroine, |
| | cocaine, marijuana |
| CVA | cerebrovascular accident |
| TIA | transient ischemic attack; also called RIND, reversible |
| | ischemic neurologic deficit |
| S/p CEA | status post carotid endarterectomy |
| NOS | not otherwise specified |
| H/O | history of, i.e. having had at least one episode |
| S/P | states post, i.e. having had |

The ACE D/D genotype declines in frequency from Western Africa (35-45% among Nigerian “control” individuals without disease) to African Americans (33% among St. Louis “controls”) to Caucasians of Western Europe and the US (25%). Although first associated with ASCAD among Western Europeans, the ACE D/D genotype nevertheless appears to have conferred a selective advantage, perhaps related to thermotolerance (Moskowitz, D W. Hypertension, thermotolerance, and the “African gene”: a hypothesis. Clinical and Experimental Hypertension: Part A. Theory and Practice 18(1):1-19, 1996.). The ACE D/D genotype is increased in frequency among African Americans carrying the hemoglobin S allele, which is felt to have been selected for the protection it confers against malaria. Finding the D/D genotype frequency increased in the same group suggests that it may have been selected for independently, as well.

The ACE D/D genotype is associated with a two-fold higher amount of plasma membrane ACE activity than the I/I genotype (and a 50% higher activity than the I/D genotype). The substrate for ACE, angiotensin I, is present at concentrations below the Km for the enzyme) in is the linear portion of the Michaelis-Menten curve. Just as an increase in substrate concentration would lead to a higher rate of product formation, so an increase in amount of enzyme would also result in an increase in the rate of angiotensin II produced. Thus, individuals with the ACE D/D genotype are expected to have twice the rate of angiotensin II production in their tissues.

Several diseases were previously thought to arise from excessive tissue concentrations of angiotensin II, such as essential hypertension and renal failure. Table 1 illustrates that many diseases are associated with an odds ratio of more than 1.0 for the ACD D/D genotype, ie many diseases are associated with excessive tissue angiotensin II production. Traditionally, odds ratios of 2.0 are felt to be “biologically significant.” For example, the odds ratio for the association of serum total cholesterol concentrations above 200 mg/dl with ASCAD is 1.7. Table 1 illustrates that a number of diseases are associated with such a large odds ratio (OR) for the ACE D/D genotype (e.g. Bipolar Affective Disorder, OR 3.78 in African American men, OR 2.33 in American Caucasian men), many of which, like Bipolar Affective Disorder, have not previously been thought to be the result of excessive tissue (including CNS) angiotensin II.

However, diseases with lower odds ratios (closer to 1.0) may also respond dramatically to treatment with an ACE inhibitor (see Tables 2ff.), thus suggesting a role for excessive tissue angiotensin II in their pathogenesis. The lack of an impressive odds ratio, coupled with a dramatic clinical response, suggests that ACE is one of a large number of interacting genes in disease pathogenesis, but that it acts so early in the disease pathway that it has an amplified effect. Disease pathways, like enzymatic pathways in general, are known to behave like “cascades”: flux through later steps is much larger than through earlier steps in the pathway.

The mechanism of action is to inhibit ACE activity, resulting in reduced angiotensin II synthesis and reduced metabolism of some vasodilating kinins. Angiotensin II constricts arterioles and stimulates aldosterone release from the adrenal cortex, which in turn stimulates Na⁺ reabsorption in the kidney. At least part of their beneficial effect is mediated through vasodilatation. Intravenous infusion of vasodilators such as nitroprusside or glyceryl trinitrate improves haemodynamics in heart failure. This effect can be sustained with hydralazine and isosorbide dinitrate given orally. This drug combination also improves survival in heart failure, suggesting that improvement in haemodynamics is at least in part responsible. However, the improvement in survival with ACE inhibitors is somewhat greater than with other vasodilators, suggesting that other mechanisms may play a part.

One mechanism is a beneficial effect on electrolyte and water balance. While vasodilators tend to increase salt and water retention, ACE inhibitors facilitate salt and water excretion by complex effects on the kidney. These effects include the attenuation of secondary hyperaldosteronism with a reduction in mineralocorticoid-stimulated sodium reabsorption. ACE inhibitors also inhibit angiotensin-mediated thirst by an action on the hypothalamus. The attenuation of aldosterone effects reduces any tendency to hypokalaemia, and this may contribute to the antiarrhythmic effect of ACE inhibitors.

Another mechanism involves the favorable effects of ACE inhibitors on the adverse neurohumoral profile which accompanies heart failure. In addition to activation of the renin-angiotensin-aldosterone system, heart failure activates several other neurohumoral systems. The increased sympathetic nerve activity and increased secretion of adrenal catecholamines probably contribute to the high incidence of malignant ventricular arrhythmias and sudden death in heart failure. ACE inhibitors produce major reductions in sympathetic nerve activity and plasma levels of catecholamines.

B. Treatment of Specific Diseases/Disorders

1. Treatment of Asymptomatic Left Ventricular Dysfunction

Heart failure is a progressive process, during which the heart undergoes major changes. For example, the patient with asymptomatic left ventricular dysfunction (This is defined as the presence of a left ventricular ejection fraction of <40-45%, in the absence of symptoms or signs of heart failure.) early post-infarction will probably have only relatively minor chamber enlargement. By the time this patient develops clinical heart failure, the heart will have enlarged substantially. This process is called ‘remodelling ’ and involves apoptosis. Echocardiographic data suggest that ACE inhibitors would prevent this process of remodelling, thereby reducing the development of heart failure and the incidence of death, highlighting yet another mechanism of action for the beneficial effects of ACE inhibitors. Most of these patients are identified following acute myocardial infarction. These patients can be considered to have asymptomatic or ‘pre-clinical ’ heart failure. Most patients have reduced effort tolerance on formal stress testing, and many will progress to overt heart failure with time. ACE inhibitors started within 1-2 weeks of the infarction improve survival, reduce the chance of developing overt heart failure and reduce the need for hospitalization.

2. Treatment of Nephropathy in Non-insulin dependent Diabetics

Angiotensin-converting enzyme (ACE) inhibitor therapy appears to be the most promising approach to slowing the development and progression of nephropathy in patients with type 2 diabetes (formerly known as non-insulin-dependent diabetes). Current recommendations for identifying early diabetic nephropathy by screening for microalbuminuria are not widely followed because the test is not uniformly available. Most screening involves testing for gross proteinuria with a dipstick or urinalysis. Many patients who might benefit from ACE inhibitor therapy do not receive it because some physicians are unaware of this clinical use for ACE inhibitors. Treating all patients with diabetes might be simpler, but the side effects associated with ACE inhibitors may affect compliance.

3. Treatment to delay progression of renal failure due to hypertension or type II NIDDM.

I. Maximize quinapril before adding any additional anti-hypertensive agent.

Inhibit tissue ACE >95%

A. Use a hydrophobic ACE inhibitor which penetrates both active sites of the enzyme. Hydrophilic ACE inhibitors such as enalapril inhibit only 50% of enzyme activity, whereas an equal amount (5 mg) of a hydrophobic ACE inhibitor such as ramipril inhibits greater than 95% of enzyme activity. Quinapril is even more hydrophobic than ramipril (ie its octanol: water partition coefficient is higher).

Practically, this means:

1. Increase quinapril (ACCUPRIL) to a maximum dose of 1 mg/pound (or 2 mg/kg) actual body weight. Actual body weight is used rather than ideal body weight because quinapril is hydrophobic and distributes into adipose tissue.

2. Use 110-120/70 mm Hg as the target blood pressure. The goal is to use the maximum dose of quinapril on everyone, so blood pressure above 105 mm Hg should be viewed as an opportunity to add more quinapril.

3. Quinapril is given in two doses: half at bed-time, and half in the morning. Thus, an 80 kg man will take 80 mg quinapril twice a day.

4. A 70 kg man (or woman) would also take 80 mg (2×40 mg tablets) twice a day, for a little more than 2 mg/kg/day.

B. Measure leukocyte plasma membrane ACE activity as a surrogate for tissue (eg endothelial cell) ACE activity. A suitable assay is given in Petrov, et al. Am J Hypertens 13(5 Pt 1):535-9 (2000), the teachings of which are incorporated herein.

C. Titrate up the dose of hydrophobic ACE inhibitor until leukocyte plasma membrane ACE activity is inhibited by greater than 95%. Based on the dose of quinapril required to achieve maximal lowering of blood pressure in rats, for example, this may require a dose as high as 3-10 mg/kg/day.

D. Because of a biological feedback loop involving angiotensin II and ACE gene activity, inhibition of ACE may result in increased transcription of the ACE gene [King, et al. Am J Physiol 263(4 Pt 1):C743-9 (1992)]. Therapeutically, the effect of this feedback loop will be to increase the dose of ACE inhibitor required to maintain greater than 95% inhibition of enzyme activity. Thus, serial leukocyte plasma membrane ACE activity determinations will be required, perhaps every 3 months for an outpatient.

E. Protect against hyperkalemia using Florinef (fludrocortisone acetate).

F. Clinic visits should be frequent enough (every 2-6 weeks) to rapidly reach the maximum quinapril dose within 3 months.

II. Control lipids, absolute blood pressure, heart rate, smoking.

A. Lipids must also be vigorously controlled. LDL-cholesterol must be lowered below 100 mg/dl. For this, use a ‘statin’ rather than dietary therapy, because it is several-fold more effective and much faster, as well as being less difficult for the patient (thus ensuring higher compliance). The current best HMG-CoA reductase inhibitor is ATORVASTATIN (LIPITOR), which lowers LDL-cholesterol as well as triglycerides. Triglycerides should be lowered below 200 mg/dl. Slow-release niacin (e.g. fuduracin) is used (500-2,000 mg every morning) to further lower serum triglycrides.

B. Do the above steps quite quickly. Atherosclerosis has been progressing for at least 20 years before a patient with a serum creatinine of 2.0 mg/dl is seen. The goal is to achieve regression. This means getting control of the situation in a very short time, over a period of weeks, not years.

C. For blood pressure control, after quinapril has been maximized, add a long-acting calcium channel blocker, such as NIFEDIPINE GITS, and maximize the dose quickly to 120 mg b.i.d as done with the ACE inhibitor. The patient may require furosemide (LASIX) if he or she develops pedal edema on NIFEDIPINE GITS. This will also help with control of serum potassium concentration. Then add MINOXIDIL at 2.5-5 mg/day, doubling the dose at each clinic visit until the blood pressure is at goal (120/70 mm Hg or less).

D. If the patient develops a pulse above 75 beats per min, add a beta-blocker to keep the heart rate at 55-60 beats per minute. Preferred beta-blockers are a cardioselective beta-1 blocker, such as metoprolol (LOPRESSOR) at 50-100 mg p.o. b.i.d. or atenolol at 25-100 mg per day. Cardioselective beta-1 blockers are preferred, especially for patients with emphysema, e.g. Bisoprolol (ZEBETA) up to a dose of 20 mg/day.

The goal is to keep the heart rate slow due to the added shear stress, and presumably activation of angiotensin I-converting enzyme (ACE), seen with faster heart rates (greater dp/dt's). Long-term epidemiologic studies show that heart rate is positively correlated with incidence of stroke, for example. A cardioselective beta-blocker is used, knowing that it may force the use of a higher dose of atorvastatin, for example, to control LDL-cholesterol, and a higher dose of FLORINEF to control hyperkalemia.

E. The patient should also be encouraged to stop smoking by using nicotine patches, since cigarette smoking is a potent contributor to atherosclerosis. A recommended schedule uses 24 hr patches, as follows: a 21 mg/day patch for 4 weeks, then a 14 mg/day patch for 2-4 weeks, the a 7 mg/day patch for 2-4 weeks.

The same protocol can be used to delay the progression of atherosclerotic peripheral vascular disease.

4. Treatment to Delay the Progression of Emphysema.

1. Begin RAMIPRIL 2.5 mg once a day (at bed-time). Increase RAMIPRIL as needed simply to keep the blood pressure at or below 120/70 mm Hg. RAMIPRIL will make the systemic blood pressure actually increase, as pulmonary hypertension is reduced and left ventricular stroke volume increases. 2. There is no upper limit to how much ramipril is used for emphysema. An appropriate dose for a patient with emphysema can be 400 mg RAMIPRIL twice a day. In one patient currently on this regime, this represents an increase from 100 mg twice a day four years ago.

5. Treatment to Decrease Cardiovascular Mortality in End-stage Renal Disease (Dialysis) Patients.

RAMIPRIL up to a maximum dose of 0.5 mg/kg/day, or Quinapril to a dose of 2 mg (kg/day), and FLORINEF to prevent hyperkalemia. Lowering the dialysate K+ to 1 mEq/liter may also be required to control serum potassium. Caution: RAMIPRIL may cause hypoglycemia, even in dialysis patients who do not have diabetes.

6. Treatment to Decrease All-causes Mortality in a Large Patient Population.

Based on the outcomes data for 4 diseases (out of the more than 40 diseases associated with an odds ratio of ≧1 with the ACE D/D genotype), an adequate (i.e. tissue-inhibitory) dose of a hydrophobic ACE inhibitor would be effective in delaying the onset or progression of all of the diseases listed in Table 1 except:

| Disease | Black men | Odds Ratio | White men | Odds Ratio |
|---|---|---|---|---|
| Asthma | | | 2 | 0.60 |
| Gall stones | 1 | 0.60 | | |
| Diverticulitis | | | 2 | 0.61 |
| Inguinal hernia | 1 | 0.63 | | |

Given the demonstrated success of the HOPE trial (NEJM January 2000) in preventing new cases of NIDDM, ramipril would be a good choice. So would another hydrophobic ACE inhibitor such as quinapril. However, the target dose for maximally effective disease prevention should be a ramipril dose of 0.5 mg/kg/day, (or quinapril 2 mg/kg/day), not merely 10 mg ramipril/day, as in the HOPE trial. Indeed, the best approach would be to attempt to inhibit tissue ACE by greater than 95% in each patient, as in Protocol 1, above. RAMIPRIL is used to treat any blood pressure over 110/70 mm Hg. In patients with any family history of cardiovascular disease or cancer, RAMIPRIL is administered to any patient having a blood pressure above 105-110 mm Hg systolic or 60 mm Hg diastolic. Other hydrophobic ACE inhibitors, such as fosinopril, benazepril, captopril and the like, may substitute for ramipril or quinapril.

Despite reports in the literature, experience with 1,000 male black and white hypertension and renal failure patients between 1994-97 revealed no adverse effects of lowering the blood pressure to 105-110 mm Hg, even in patients with chronic blood pressures of 180/110 mm Hg previously. No “J-point” in the curve of clinical events vs. blood pressure was observed.

ACE inhibitors are fetopathic and contraindicated in pregnancy. Women of childbearing age taking RAMIPRIL should use contraception (preferably a barrier method rather than oral contraceptives, to further decrease side effects such as hypercoagulability and hyperlipoidemia) as well as undergo pregnancy testing within the first two weeks after a missed menstrual period. RAMIPRIL can be used on every normotensive male patient over the age of 25, and on every hypertensive patient regardless of age.

QUINAPRIL seems to have been superior to RAMIPRIL for slowing down kidney failure, so QUINAPRIL is preferred over RAMIPRIL for patients with any renal disease, or family history of renal disease.

There was no added benefit of LOSARTAN 50 mg/day when added to QUINAPRIL in patients with kidney failure. Therefore the use of an angiotensin receptor blocker (ARB) either alone or in combination with an ACE inhibitor is not preferred. The only indication for adding an ARB to an ACE inhibitor would be in the case of serum hypokalemia. The only reason to use an ARB at all would be in a patient who develops a severe reaction to an ACE inhibitor, e.g. angioedema (approximately 1%), disabling cough (approximately 5-10%), or leukopenia (approximately 1/10,000 patients on ACE inhibitors). Many patients with a dry cough due to RAMIPRIL or QUINAPRIL can tolerate the cough with a cough suppressant (e.g. ROBITUSSIN DM, 1 teaspoon q.i.d).

Additional maneuvers to decrease the synthesis of tissue angiotensin II, e.g. by inhibition of chymase in tissues in which non-ACE production of angiotensin II is significant, or inhibition of the downstream effects of angiotensin II (e.g. by inhibition of TGF-beta or endothelin) or antagonism of angiotensin II (by stimulation of nitric oxide production, e.g. by oral supplementation with a substrate for nitric oxide synthase such as L-arginine) are expected to add to the effectiveness of the above regimen.

Serum potassium concentration can be decreased in patients for whom ACE inhibition is indicated.

I. Control of Hyperkalemia by Exploiting the Renin-angiotensin System

Quinapril (ACCUPRIL) is indicated for prevention of chronic renal failure, among other diseases, as described above. The problem is that patients with chronic renal failure have Type IV Renal Tubular Acidosis (Type IV RTA, so-called “hyporeninemic hypoaldosteronism”) with hyperkalemia as a result of their renal insufficiency. This condition is only exacerbated by angiotensin I-converting enzyme (ACE) inhibitors such as quinapril, since ACE inhibitors block the production of angiotensin II, which is a major stimulus for the medullary adrenal gland to synthesize aldosterone. Aldosterone is the major hormone responsible for Na+ for K+ exchange in the distal nephron. In the absence of aldosterone, potassium is not excreted into the urine, and its concentration in the bloodstream increases.

Serum K+ concentration is tightly controlled, with the normal level being accepted as 3.5-5 mEq/l. Variation outside this range can have catastrophic consequences; the more acute the change, the less variation is tolerated, since compensatory mechanisms will not have had time for maximum deployment. For example, it is generally agreed that, cardiac conduction abnormalities, including total cardiac standstill, occur above a serum potassium level of 7 mEq/l, even when it arises over weeks to months, i.e. chronically. In general, patients with end-stage renal disease are maintained at a serum potassium concentration at or below 5.3 mEq/l.

As described above, fludrocortisone acetate (FLORINEF) can be used to control serum K+ concentration. Data were provided showing that the use of up to 0.1 mg daily of Florinef could control serum potassium in patients with chronic renal failure who were given high doses of an ACE inhibitor (e.g. 2 mg/kg total body weight/day quinapril; Moskowitz, From Pharmacogenomics to Improved Patient Outcomes: Angiotensin I-Converting Enzyme as an Example. Diabetes Technology & Therapeutics. 4 (4): 519-531, 2002).

Based on this data, the combination of any ACE inhibitor (e.g. hydrophilic ACE inhibitors such as captopril, enalapril, lisinopril, etc., or hydrophobic ACE inhibitors such as ramipril, benazepril, or quinapril [ACCUPRIL]), in any fixed dose, with fludrocortisone acetate is of particular usefulness in any patient with an indication for an ACE inhibitor who has a serum K+ concentration above 4.5 mEq/l before initial dosing.

For example, a 100 kg male patient with a serum creatinine of 3.0 mg/dl due to diabetic nephropathy has a serum potassium concentration of 5.2 mEq/l and a blood pressure of 160/100. Traditionally, this patient would not be considered a candidate for ACE inhibition because his Type IV RTA will only be exacerbated by addition of an ACE inhibitor. The patient can be given an ACE inhibitor and Florinef separately. Alternatively, the patient can be given a combination drug, with the following caveats:

The total amount of Florinef should ideally not exceed 0.1 mg/day.

No diuretic is required, i.e. no fluid retention due to Florinef occurs, when Florinef is used at 0.1 mg a day but less than daily dosing per week. That is, the “Florinef holiday” can be as short as two consecutive days a week, e.g. Saturday and Sunday on a weekend.

7. Treatment of Presbycusis

ACE inhibitors can be used to treat presbycusis (“hardness of hearing”), which is common in both humans and non-human species such as dogs and cats. Presbycusis can also be mimicked by acoustic trauma to the ear, as in occupational exposures in the manufacturing, music, and aviation industries, to name just a few.

The dose of ACE inhibitor required may be at least 2 mg/kg/day quinapril (or at least 0.5 mg/kg/day ramipril) in two divided doses. A sustained release formulation may well allow for less frequent dosing. Since dogs and cats experience presbycusis, this treatment regimen is also applicable to them. An even higher dose of ACE inhibitor (up to 10 mg/kg/day quinapril, or 2.5 mg/kg/day ramipril) may be required for maximally effective prophylaxis against presbycusis or occupational hearing loss.

8. Treatment of Age-dependent Diseases

A variety of age-dependent diseases are related to the ACE enzyme. C-myc is at the start of the apoptosis pathway. C-myc activation is a result of angiotensin II signaling, and ACE is postulated to be the rate-limiting step in humans for tissue angiotensin II production. Since normal tissue loss due to apoptosis, eventually results in disease, effective tissue ACE inhibition can be used to delay or prevent disease.

Specific examples:

1. Loss of pulmonary parenchyma with aging: a consequence of apoptosis, presumably mediated by ACE activation in the pulmonary arterial circulation.

2. Loss of renal function with age: GFR declines 1% per year. This also represents apoptosis of nephrons under the continued drive of angiotensin II signaling. In the kidney, the primary source of angiotensin II is the proximal tubular brush border membrane.

3. Atherosclerosis: evolution of the atherosclerotic plaque is a hallmark of atherosclerosis. The plaque contains ACE due to the presence of T lymphocytes and macrophages in the plaque. Angiotensin II is pro-thrombotic, and stimulates proliferation of smooth muscle cells. Reactive oxygen species which are found in the plaque are the result of increased activity of T cells and macrophages. Angiotensin II is a cytokine which stimulates T cell and macrophage function, including generation of oxygen radicals.

4. Cancer: angiotensin II is a potent growth factor for a variety of cells. The presence of a constant growth stimulus is an important precondition for escape from growth control, which is what cancer represents.

5. Exercise is also an anti-aging process. The effects of exercise—vasodilation, improvement in mood, decrease in the incidence of colon cancer, among others—are mimicked by ACE inhibition. The combination of ACE inhibition and exercise should be more potent than either alone in delaying the aging process.

Aging has been linked to metabolic rate in rodents; caloric restriction prolongs lifespan. Metabolic rate scales linearly with glomerular filtration rate (GFR), and appears to be the major determinant of GFR. GFR, and glomerulo-tubular balance[G-T balance], are regulated by angiotensin II. ACE in the brush border membrane of proximal tubular cells, especially the S1 segment, appears to be the mechanosensor for G-T balance, and the rate-limiting step for All production.

The characteristics of aging, such as apoptosis of organs, neoplasia, and appearance of other age-related diseases, all appear to be related to the ACE gene by molecular epidemiologic data. Intermediate biochemical surrogates of aging, such as generation of reactive oxygen species can also be laid at the feet of angiotensin II. If activation of endothelial and tissue ACE by turbulent flow in the circulation is the cause of aging, then inhibition of ACE and slowing of the heart-rate should decrease aging. Tissue ACE can be inhibited effectively with at least 2 mg/kg/day quinapril; higher doses may be even more effective, e.g. 3-10 mg/kg/day. Heart rate can be slowed with a beta-blocker to the desired level of 60 beats per min in humans.

C. Determining Optimal Ace Inhibitor Dosages

The optimal dosage can be determined using a protocol as described below, or one based on such a study.

1. Dosage in Treatment of Hypertensive Nephropathy

For example, a short-term dose-response study over a period of 8 weeks can be used to determine the optimal dose in microalbuminuria. For hypertensive nephropathy (creatinine <1.6 mg/dl), a minimum of 6 (maximum of 20) male patients and the same number of female patients are randomized to each of 4 treatment groups: placebo, 2 mg/kg/day, 3 mg/kg/day, or 4 mg/kg/day quinapril. The quinapril dose is calculated on the basis of total body weight; the total dose is divided into two doses, one at bedtime, and one upon arising in the morning.

Using the minimum cell size (6), a total of (6+6)×4=48 patients will be required. The larger sample size (20) will require a total of (20+20)×4=160 patients. The advantage of using the larger sample size of 20 per gender is that patients could also be ACE I/D genotyped. Since about 30% of Caucasians with hypertensive nephropathy are ACE D/D, a cell size of 20 should yield 6 patients who are D/D. (Half, or 10 out of 20, will be I/D, and 20%, or 4 out of 20, will be I/I). This additional data could test the hypothesis that ACE D/D patients will require a higher dose of quinapril to achieve the same decrease in albumin excretion as ACE I/D and I/I patients.

Weeks 1 and 2: obtain at least two measurements of 24 hr albumin excretion rate on each patient.

Week 3: randomize to one of the 4 treatment groups and begin titrating up the dose of quinapril. Target BP is 105-120 mm Hg systolic, <75 mm Hg diastolic; target heart rate is 55-65 beats per minute; target LDL is <100 mg/dl using atorvastatin (LIPITOR).

Weeks 4-6: continue titrating up quinapril, increasing dose twice a week (e.g. Mondays and Fridays).

Weeks 7 and 8: repeat at least two measurements of 24 hr albumin excretion rate on each patient.

Conduct data analysis using paired t-test using average of the 2 albumin excretion rates for weeks 1 and 2, compared with the average for weeks 7 and 8.

The same regime can be used for diabetic nephropathy (creatinine <1.6 mg/dl; patients with NIDDM).

Medium-term studies can then be used to demonstrate efficacy of higher than conventional doses of quinapril for common diseases, as f

2. Dosage For Treatment of Chronic Renal Failure (CRF) due to Hypertension

Using the optimal dose established in Study IA (microalbuminuria in hypertension), use each patient as their own control. Collect 25 men and 25 women with hypertensive nephropathy in each of three categories of renal function, for a total of 150 patients:

(i) Serum creatinine <2 mg/dl (estimated creatinine clearance >50 ml/min)

(ii) Serum creatinine >2 mg/dl but <3 mg/dl (creatinine clearance between 30 and 50 ml/min)

(iii) Serum creatinine >3 mg/dl (creatinine clearance <30 ml/min).

Month 1: On current medication, collect serum creatinines at time 0, week 2, and week 4 to establish a baseline 1/creatinine vs. time curve. Incorporate any previous serum creatinine values available.

Months 2 and 3: Titrate each patient to the target quinapril dose established as the optimal dose in Study IA. Control LDL <100 mg/dl, pulse approximately 60/min, and BP <120/75 mm Hg as described in Study IA above.

Months 4-6: Obtain serum creatinine every 2-4 weeks. Genotype patients for the ACE I/D polymorphism.

Using each patient as their own control, determine the slope of 1/creatinine vs. time before and during high-dose quinapril treatment, using linear regression. Perform paired t-test to determine if the slopes are significantly different at the p<0.05 level.

Perform a sub-group analysis based on ACE I/D genotype to see if the D allele influences response to treatment. (If this latter analysis is omitted, only 6-8 patients are required for each serum creatinine level, for a total of 36-48 total patients).

The same protocol can be used to optimize dosage for diabetic nephrophathy (due to NIDDM).

3. Treatment of ASPVD due to HTN.

6-8 male patients with claudication due to hypertension are given high-dose quinapril, and their response is measured using an exercise treadmill (time to first symptom of claudication). An equal number of male patients are randomized to receive placebo rather than quinapril.

Month 1: Establish baseline exercise tolerance (time to first symptom of claudication on a treadmill at 2 miles per hour, 0% incline) on at least two separate occasions.

Months 2 and 3: Titrate each patient's quinapril dose to the optimal discovered for short-term lowering of albumin excretion rate in hypertensive patients (Study IA above). Bring each patient's BP, LDL, and pulse to target values. The placebo group is treated with a calcium channel blocker, e.g. amlodipine.

Month 5: Repeat exercise tolerance test×2, using treadmill (time to first symptom of claudication).

Use average of the two times to claudication for each time period. Paired t-test, with intention to treat, comparing “before” and “during” high-dose quinapril treatment. To control for the effects of bringing BP, LDL and pulse to target values, a placebo group is included.

4. Treatment of ASPVD due to NIDDM

As for Study II C, using patients with claudication and NIDDM.

5. Treatment of COPD

Patients with severe COPD due to cigarette abuse (at least 1.5 pack per day) are recruited: 8-12 men with FEV-1 <1 liter, requiring at least 1 liter/min oxygen by nasal prongs for 24 hr/day. Exercise tolerance on a treadmill or bicycle is used to quantify the response, if any, to quinapril. An equal number are randomized to receive placebo.

Month 1: At least two exercise treadmill (or bicycle) exercise tests, stopping at “moderate” shortness of breath. (If “10”, is “choking to death” and “1” is baseline, stop at “5”). Objectively, stop when heart rate reaches 120 beats per min.

Month 2: Titrate quinapril to a final BP of 110-120/75. Note that quinapril will result in an increase in systemic BP, since it decreases pulmonary hypertension.

Month 4: Repeat exercise treadmill test×2.

Paired t-test, using the average of the two times to end-point. If 25 men are used in each category (quinapril and placebo), then an estimate of the effect of the D allele on response to treatment can be made.

Method

Ramipril or quinapril, both hydrophobic ACE inhibitors, can be used.

The dosage is titrated up in each dialysis patient with careful attention to serum potassium concentration. In other words, for hemodialysis patients, serum K+ concentration is checked at the beginning of dialysis within 48 hr after the first dose of ACE inhibitor.

6. Determination of Dosages for Treatment of Other Diseases

For each additional disease, such as glomerulonephritis (e.g. Focal segmental glomerulosclerosis, FSGS) or CHF, a dose-response curve can be established for each patient. A quantifiable index of the patient's disease is followed, e.g. proteinuria in FSGS, or exercise tolerance in CHF (e.g. stairs climbed before the onset of overwhelming dyspnea).

The patient is placed on a starting dose of quinapril of 2 mg/kg/day in 2 divided doses, and followed for a period of about 2 months. If there is no change in the quantifiable variable from baseline, then the quinapril dose is increased to 3 mg/kg/day, and the patient is followed for an additional 2 months. If there is still no change, then the dose is increased to 4 mg/kg/day, if allowed by the patient's blood pressure.

An alternative is to measure leukocyte plasma membrane ACE activity, and settle on a dose of quinapril which inhibits 100% of white cell ACE activity, used as a surrogate for tissue ACE activity.

In addition to the diseases listed above, common solid tumors in women, e.g. breast, endometrial, and ovarian cancer, are also associated with the ACE deletion/deletion (D/D) genotype, and will be ameliorated by use of quinapril (or ramipril) early enough in the course of these diseases.

For maximal effectiveness, quinapril (or ramipril) should be started at as high a dose as tolerated at the earliest possible time in the course of disease. There is abundant evidence that atherosclerosis begins in childhood. For example, autopsies of healthy 18 year olds during the Korean War showed fatty streaks in the aorta, the hallmark of macrovascular disease.

If ACE is indeed a mechanotransducer, rising blood pressure is expected to trigger ACE activity, especially in areas of turbulent flow. The molecule of ACE does not extend sufficiently far into the blood stream to enter the regime of convective fluid transport; rather, it is small enough (<300 Angstroms, or 30 nm) to remain in the unstirred layer at the cell surface where only diffusion operates. It is activated by shear stress, therefore, only in regions of turbulent flow. In such flow regimes, the velocity vector of blood flow has a component, momentarily at least, oriented at right angles to the cell surface.

As a result of ACE's activation by turbulent flow, by its involvement in most common serious diseases (atherosclerotic complications, psychiatric diseases, and solid cancers), systemic blood pressure can be used as the index for when to start prophylactic quinapril. Quinapril can be started whenever systolic blood pressure is found to be >110 mm Hg, since the correlation between blood pressure and complications of hypertension has no threshold value. (Nor is there a “J-point” implying a danger of lowering blood pressure below 110-120 mm Hg to as low as 95 mm Hg, say, when orthostatic symptoms begin to supervene.) Indeed, the average systolic blood pressure for a healthy 18 year old is 90 mm Hg, and the goal of this therapy is to keep people at a state of health of the average 18 year old.

The short-term goals of therapy are:

1. Inhibit endothelial cell membrane ACE by 100%. A surrogate clinical test is leukocyte membrane ACE activity.

2. Keep systolic blood pressure at 95-105 mm Hg. Diastolic blood pressure must be kept below 80 mm Hg.

3. Keep the pulse at 60 beats per minute to minimize turbulent flow.

The long-term goals of therapy are:

1. Prevent complications of systemic hypertension.

2. Decrease insulin resistance, so as to decrease the secretory load on the endocrine pancreas.

3. Decrease angiotensin II (and compensatory NO) levels, so as to decrease activation of c-myc, and the caspase pathway of apoptosis. This will decrease the push for pancreatic apoptosis in particular, and hence development of NIDDM. Use of ramipril in the HOPE study has already shown prevention of new cases of NIDDM using a hydrophobic ACE inhibitor (NEJM Jan. 21, 2000).

4. By decreasing activation of c-myc, decrease activity of the cell growth and proliferation pathways. This will decrease the push for development of cancerous cells which have escaped growth control.

5. Decrease stimulation of catecholamine pathways in the CNS, leading to less release of catecholamines at nerve synapses and less resultant depletion of catecholamine stores. Indeed, use of quinapril by 12,000 patients was associated with an elevation of mood in 20% of patients (Parke-Davis, personal communication). This should decrease the progression of psychiatric diseases associated with the ACE D/D genotype with odds ratios of >1 such as bipolar affective disorder, depression, anxiety, panic disorder, and schizophrenia.

Beginning quinapril when systemic blood pressure reaches 110 mm Hg can be a useful guide for the prevention of cardiovascular diseases including NIDDM. Quinapril is then slowly (q 2 weeks) titrated upwards towards the target dose of 2 mg/kg/day, as limited by the patient's blood pressure (usually >100 mm Hg) and presence of orthostatic lightheadedness. But psychiatric diseases such as schizophrenia often occur before age 20, in the setting of normal systemic blood pressure. Similarly, many patients with early onset of solid tumors in their 40's for example, still have normal systemic blood pressure. Finally, not all patients with complications of hypertension such as chronic renal failure or stroke have elevated systemic blood pressure. For example, there are patients with hypertensive nephrosclerosis on renal biopsy but whose systemic blood pressure never exceeds 120/80, which is now considered well below the target of antihypertensive therapy. Such patients have “chronic renal failure of insidious onset” (Hospital Practice, circa 1997). An example is Patient HK, whose, creatinine decreased from 4.5 m/dl to 1.7 mg/dl on 20 mg/day quinapril, given hs, with a resultant blood pressure of ˜95 mm Hg systolic but no orthostatic symptoms or signs. Presumably, in these patients there is a much higher local tissue effect of ACE than there is on systemic blood pressure.

It is postulated that tissues are equipped with different downstream pathways for the operation of angiotensin II (and bradykinin). Systemic blood pressure is the product of cardiac output and systemic vascular resistance (BP=CO×SVR); SVR can be kept low by overexpression of vasodilatory compounds such as induction of nitric oxide synthesis. Progression of renal disease may either not involve nitric oxide, or the enzyme responsible for NO production in arterioles (presumably ecNOS, or NOS 3) may be under different control in the kidney such that NO is overproduced in arterioles but not in kidney parenchyma.

Therefore, the indication for beginning patients on quinapril or ramipril in the absence of a systemic blood pressure above 105-110 mm Hg systolic should be a familial risk of disease, or else the earliest manifestation of disease. Once the individual nucleotide polymorphisms have been identified for all diseases, then a person's risk of future diseases will be able to be foretold with accuracy. Until that time, the occurrence of a particular disease within the patient's family, or the earliest symptom or sign consistent with a chronic disease (e.g. microalbuminuria despite a normal serum creatinine as a predictor of chronic renal failure; emotional lability in a teenager with a family history of mental illness) should serve as a sufficient indication to start the patient on prophylactic therapy with quinapril.

Obviously, all current precautions using ACE inhibitors should be observed. In particular, women of child-bearing age should be given birth control measures (oral contraceptive pill or barrier methods); in the event of pregnancy, the ACE inhibitor (quinapril or ramipril) should be discontinued immediately. It is safe, however, for nursing mothers to resume prophylactic quinapril, however.

The goal of tissue ACE inhibition in this patient population is to prevent vascular ACE-mediated production of angiotensin II, and downstream potent growth factors for fibroblasts and vascular smooth muscle cells such as TGF-beta1, endothelin, IGF-I. The result will be delay in the accelerated atherosclerosis seen commonly in ESRD patients. Clinically, this will take the following forms:

1. A delay in progression to vascular calcification of the extremities (so-called calciphylaxis, which is now primarily attributed to PTH and vitamin D excess).

2. A decline in the incidence of graft stenosis, graft thrombosis, and similar vascular access problems that account for the major morbidity of patients undergoing hemodialysis.

3. A delay in the progression of atherosclerotic coronary artery disease, which is the major cause of mortality in ESRD patients. For example, a patient with NIDDM and ESRD has a 37-fold higher risk of myocardial infarction than the population average.

D. Non-human (Including Veterinary) Use of ACE Inhibitors

Renal failure, especially in small and large cats, including cheetahs in captivity; degenerative joint disease in dogs and horses; and neoplasia in dogs and household pet cats are major causes of morbidity and mortality. In a human population, all of these diseases were associated with the ACE deletion/deletion (D/D) genotype. The ACE I/D polymorphism does not exist in all mammals, however, so the same polymorphism cannot be used to show an association of the ACE gene with these diseases in non-human species. However, there are a number of linked polymorphisms (17 are known), so perhaps one or more of these can be used to show the association of the ACE gene with such diseases in non-human species. Comparative medical genomics suggests that genes associated with human diseases will also be associated with similar diseases in related species.

The ancestral, human testicular form of ACE consists of a single active site contained in a protein encoded by 13 exons. This form of the enzyme, a general dipeptidase, is present in all species, including bacteria, where it is homologous to thermolysin. A highly homologous form of human testicular ACE exists in the roundworm, C. elegans, and in Drosophila. Thus, there is virtually no limit to the species which may benefit from ACE inhibition to increase their longevity, apart from plants. Any commercial animal species useful to humans is a candidate for therapy with adequate, tissue-inhibitory doses of a hydrophobic ACE inhibitor.

Tissue ACE inhibition should also prolong the lifespan of fish, many of which have commercial uses, and some of which are grown in farms. Many fish are kept as pets, especially expensive, hard-to-obtain coral reef fish. A hydrophobic ACE inhibitor such as quinapril or ramipril may be included in the fish food in order to prolong the fish's lifespan. Since ACE is present in all animal species, and even prokaryotes, and since ACE-mediated generation of angiotensin II, is one of the main reasons why all species age, including Drosophila and C. elegans and other well-studied animal models of aging, then it should be possible to extend the lifespan of all living animals (and perhaps even unicellular organisms such as bacteria and yeast) using effective ACE inhibition. Similar ideal values can easily be discovered by routine experimentation for each species, if not already known.

### II. ACE Inhibitors and Formulations Thereof

ACE inhibitors are now the most widely prescribed drug for hypertension and are used as the first line treatment and include drugs such as Captopril, Enalapril, Lisinopril, Alacepril, Benazepril, Cilazapril, Perindopril, Quinapril, Ramipril, Zofenopril. The efficacy of these compunds can be enhanced with β-adrenergic agonists or diuretics.

A. High Dosage Formulations

ACE inhibitor's have been in use to treat patients with chronic renal failure since the early 1980s, with clear demonstrations of their anti-proteinuric effect. Yet the rate of progression of renal failure was not delayed at all for African Americans (JAMA 1992, MRFIT study), although they have a 4-6 fold higher incidence of end-stage renal disease (ESRD) than Caucasians, whose rate of progression was halted by the same physicians using the same medications.

The data in Table I showing an odds ratios above 1.0 for ESRD due to hypertension or NIDDM in 1993-4 reinforces the suggestion that ACE, and its product, angiotensin II, may cause ESRD. If ACE is indeed an early step in pathogenesis of ESRD, as nephrologists had long suspected and as confirmed by molecular epidemiologic data herein, and if ACE inhibitors were already being used for patients with little effect, then the only explanation had to be that the ACE inhibitor's were not being used effectively. Since tissue parenchymal damage is thought to involve tissue ACE (ie present primarily on endothelial cells, and in specialized cells such as the brush border membrane of renal proximal tubular cells), tissue ACE needs to be inhibited effectively.

Effective inhibition of tissue ACE requires both the right ACE inhibitor (a hydrophobic drug such as rampril or quinapril that can penetrate both active sites of the enzyme, not a hydrophilic drug such as captopril or enalapril which binds to only one, solvent-accessible, active site in the enzyme) and an adequate dose.

In practice, physicians use quinapril at a dose of 40 to 80 mg/day, i.e. up to 1 mg/kg per day for a “typical” 80 kg patient. In rats, maximum blood pressure lowering (an index of maximum tissue inhibition; there are no studies yet showing what dose of ACE inhibitor is required for tissue ACE inhibition, only serum ACE in rats or humans) did not occur until a dose of 3 mg/kg IV quinapril. In other words, a clear reason for failure to delay progression of chronic renal failure is that too small a dose of ACE inhibitor has been used.

There are surprisingly few risks of increasing the dose of ACE inhibitor, apart from raising serum potassium. The dose-toxicity curve for quinapril, for example, is flat from 5 mg/day to 80 mg/day. Accordingly, the recommended dosage is to administer greater than 80 mg/day of an ACE inhibitor such as quinapril.

Ramipril is currently packaged as 2.5 mg, 5 mg, and 10 mg tablets. Currently, the maximum recommended dose for hypertension, as approved by the FDA, is 20 mg/day, which can be achieved with 10 mg twice a day. However, doses of at least 0.5 mg/kg total body weight/day result in improved clinical efficacy, as in patients with chronic renal failure due to hypertension or type 2 diabetes mellitus. For an average adult weighing 80 kg, then 40 mg a day ramipril is required. This can be conveniently given as a single 20 mg pill taken twice a day.

In emphysema (chronic obstructive pulmonary disease, or COPD), ramipril appears to lower pulmonary hypertension, allowing for increased delivery of blood to the left ventricle, increased cardiac output, and therefore higher systemic blood pressure. Many patients with COPD were once hypertensive, although progression of their COPD results in lowering of their systemic blood pressure due to decreased flow through the high-pressure pulmonary circulation. Quite high doses of ramipril can be required to control systemic blood pressure in these patients, e.g. 700 mg/day. Taking these quantities of ramipril is inconvenient if the largest tablet size is only 10 mg. But 7.00 mg/day can be conveniently taken as 3×100 mg plus 1×50 mg, twice a day.

Therefore, the following dosage formulations of ramipril have been developed:

20 mg

50 mg

100 mg

200 mg

Formulations have been developed for the use of tissue-inhibitory doses of hydrophobic ACE inhibitors such as ramipril and quinapril to reduce morbidity and mortality from a large number of common diseases. As described herein, use of ramipril (up to 0.5 mg/kg/day in 2 divided doses) or quinapril (up to 2 mg/kg/day) is specifically suggested for the treatment of patients with established renal failure requiring renal replacement therapy (e.g. Hemodialysis or peritoneal dialysis), i.e. Patients with end-stage renal disease (ESRD). At least twice as much enalapril is required as ramipril, since circulating ACE is inhibited 47% by 5 mg enalapril but 97% by the same dose of ramipril. For tissue ACE, the difference may be even more pronounced. The goal of therapy should be to inhibit both active sites of the enzyme, both the hydrophilic and the hydrophobic site. It is unclear how the accessibility (on and off rates) of the two active sites compares for the tissue vs. the circulating forms of ACE. There is a suggestion that the hydrophobic active site becomes occluded, resulting in a prolonged off-rate [t_{(1/2)}˜24 hr for quinapril or ramipril] compared to the hydrophilic site in the circulating form of the enzyme [t_{(1/2)}˜4 hr for enalapril]. Thus, a bio-equivalent amount of enalapril may need to contain at least twice the amount, in mg, as ramipril.

The concentration of ACE in some tissues is much higher than in others. For example, the pulmonary circulation contains more ACE than any other part of the body. As a result, amounts of ramipril up to 700 mg/day [7.3 mg/kg/day] may be necessary to control pulmonary and systemic blood pressures in patients with COPD, whereas a dose of 0.5 mg/kg/day ramipril [or 2 mg/kg/day quinapril] may be sufficient to delay the progression of chronic renal failure due to hypertension.

B. Sustained Release Formulation for ACE inhibitors

A sustained-release (SR) formulation of ACE inhibitor's is extremely useful in the delivery of the correct dose of ACE inhibitor in a convenient and safe way for each patient. The SR formulation can be in the form of a tablet or capsule, a sustained or controlled release polymeric formulation, an osmotic pump, a depo, or a gel or other implant that releases over a prolonged period of time.

There are two reasons to prefer a sustained release (SR) formulation for ACE inhibitor's: (1) to avoid high peak serum ACE inhibitor concentrations, and (2) to decrease dosing frequency for the patient's convenience.

Currently, ACE inhibitor's are used in twice-a-day dosing with good clinical effects. However, in using relatively high doses of quinapril or ramipril, or even higher doses of hydrophilic ACE inhibitor's such as enalapril, there is some danger of orthostatic hypotension if a bolus of the active, de-esterified drug (quinaprilat, ramiprilat, enalaprilat, etc.) is released into the circulation by the liver. Using the current formulation, the peak serum concentration of the parent drug (ramipril or quinapril) is reached in ≦1 hr; the peak concentration of the de-esterifed form of the drug (ramiprilat or quinaprilat) is reached in 2-4 hr. Besides decreasing the incidence of orthostatic hypotension, a SR formulation may decrease the rate of angioedema, which necessitates discontinuation of all ACE inhibitor's.

A preferred alternative is to use once-a-day dosing with a sustained-release drug. Thus, instead of dosing a 220 lb. (=100 kg) man with 100 mg quinapril twice a day, the patient would be treated with a single 200 mg quinapril sustained release (SR) tablet taken once a day. Alternatively, he could be given 100 mg SR quinapril twice a day, which would create a more constant quinaprilat serum concentration if not actually cutting down on the frequency of his dosing. As a second example, instead of giving a 100 kg man with COPD 350 mg ramipril twice a day, he would be treated with 700 mg sustained release (SR) ramipril once a day, or 350 mg SR ramipril twice a day.

Ramipril and quinapril could be formulated in 50, 100, and 200, and 500 mg SR tablets (or capsules) for convenience in arriving at the right dose with the minimum number of tablets.

C. Formulations for Treating Hyperkalemia

Hyperkalemia, which is dose-dependent, is the main reason why ACE inhibitor's are not used at adequate doses in renal failure patients. It is caused by hyporeninemic hypoaldosteronism, or so called “Type IV renal tubular acidosis (RTA).” Hyperkalemia results when production of angiotensin II, which normally stimulates the production of the kaliuretic hormone aldosterone, is decreased, e.g. by ACE inhibitor's. For reasons still not understood, chronic renal failure itself produces a Type IV RTA, which is exacerbated by ACE inhibitor's, hence leading to nephrologists' reluctance to use higher doses of ACE inhibitor for their patients with chronic renal failure. Perhaps the Type IV RTA of chronic renal failure has something to due with decreased angiotensin II production due to loss of nephrons and in particular loss of proximal tubular brush border membrane (BBM) ACE. If so, then the loss of angiotensin II must be highly compartmentalized, since progression of chronic renal failure is thought to be due to excessive angiotensin II production.

One way to rationalize this paradox is to suggest that angiotensin II produced in the lumen by proximal tubular BBM ACE acts on the distal nephron to stimulate kaliuresis, but is not involved in progression of chronic renal failure. Indeed, luminal angiotensin II may be required to counteract the effect of extra-luminal angiotensin II (derived from vascular endothelial cells within the kidney) binding to basolateral angiotensin II receptors. Perhaps luminal angiotensin II receptors are coupled to ion transport whereas basolateral angiotensin II receptors are coupled to cell growth and atrophy.

There are currently two known classes of angiotensin II receptors, AT1 and AT2. The AT1 receptor appears to mediate cell growth and apoptotic signals of angiotensin II; perhaps the AT2 isoform mediates changes in ion transport.

There are several methods to reduce the serum potassium, if necessary:

1. Decreasing the K+ concentration in the dialysate bath to 2.0, 1.5, 1.0, or even 0 mEq/l.

2. Prescribing Florinef at a dose of 0.1 mg 2-7 times per week. Despite the absence of functioning distal nephrons, the colon also is responsive to aldosterone and can perform net Na+ for K+ exchange, like the normally functioning distal nephron.

3. Under extreme circumstances, prescribing oral Kayexalate.

An adverse effect of using ACE inhibitor's in ESRD patients is the possibility of reversing the insulin-resistance of essential hypertension. In two black hemodialysis patients with ESRD due to hypertension who were prescribed ramipril 5 mg/day (a small dose), serum glucose dropped to 40 mg/dl as well as serum potassium rising to above 6 mEq/l. Hypoglycemia could be corrected by increasing glucose concentration in the dialysate or encouraging patients to eat frequent snacks.

Hyperkalemia due to Type IV RTA can be very effectively managed by replacing the absent aldosterone. This can be easily achieved using FLORINEF (fludrocortisone acetate; see Table 2).

TABLE 2
The use of FLORINEF (fludrocortisone acetate) to control
hyperkalemia due to Type IV renal tubular acidosis, such as due to
use of an ACE INHIBITOR, or due to chronic renal failure itself.

| Serum [K+], | | |
|---|---|---|
| mEq/l | Dose (in mg) | Frequency |
| 4.5-4.7 | 0.1 | once a week (Monday AM) |
| 4.8-5.0 | 0.1 | twice a week (Mon and Friday Ams) |
| 5.1-5.3 | 0.1 | three times a week (Mon, Wed, Fri Ams) |
| 5.4-5.5 | 0.1 | five times a week (Mon-Fri Ams) |
| 5.6- . . . | 0.1 | daily* |

*Daily use of FLORINEF requires daily use of a diuretic, such as furosemide 20 mg/day (for creatinine >2.5 mg/dl) or furosemide 40 mg/day (for creatinine >2.5 mg/dl), to prevent fluid retention and congestive heart failure. Clinical observation indicates that volume retention occurs with daily FLORINEF but not with less-than-daily use, even up to five times a week.

The higher the serum creatinine to start with, the higher the dose of Florinef will be needed initially. Thus, a renal failure patient with a serum creatinine of 4.5 mg/dl and a serum [K+] of 5.1 mEq/l receiving Quinapril 40 mg p.o. twice a day at the first clinic visit might need 0.1 mg Florinef 5 times a week rather than only 3 times a week because of: 1) the high serum creatinine; 2) the high dose of quinapril being used from the very beginning. The rules above are useful when patients are slowly being advanced in their quinapril, e.g. from 20 mg once a day (at bed-time) at the first clinic visit, to 20 mg twice a day at the second visit (1-2 months later), to 40 mg twice a day (1-2 months later), to 80 mg twice a day (1-2 months later).

To accomplish the above two goals, i.e. to use Florinef at less than daily dosing so as to avoid the need for a diuretic, with the resulting hyperreninemia and elevation of angiotensin II production in tissues, a combination drug can be used, to be taken twice a day (b.i.d.) so as to keep constant the serum concentration of a long-acting ACE inhibitor such as quinapril or ramipril. (NB. A combination drug involving a shorter acting ACE inhibitor such as captopril or enalapril, which has an effective half-life of 4-6 hr, would require correspondingly more pills per day, say 4 times a day (q.i.d.) with each pill containing 0.1 mg divided by 4, or 25 mcg of Florinef).

Examples of such a combination pill is given below:

Quinapril 40 mg with 0.05 mg (50 mcg) Florinef, or

Quinapril 80 mg with 0.05 mg (50 mcg) Florinef.

If the patient is to take a total dose of 2 mg/kg/day, in two divided doses, of quinapril, then he will require 2×100=200 mg/day, in 2 divided doses, or 100 mg quinapril bid. For this patient, the optimal combination pill would be Quinapril 100 mg with 0.05 (50 mcg) Florinef, so that his total dose of Florinef would be 0.1 mg/day. The patient would take a quinapril pill without Florinef (i.e. quinapril 100 mg b.i.d.) on the weekends only in order to achieve the “Florinef holiday” that would make addition of a diuretic unnecessary.

An equivalent dose of ramipril would be 0.5 mg/kg/day. The patient would therefore require 0.5×100=50 mg ramipril per day, in 2 divided doses. For this patient, the optimal combination pill would therefore be ramipril 25 mg with 0.05 mg (50 mcg) Florinef, with the same considerations as above. In other words, this patient would alternate his ramipril-Florinef combination pill with ramipril alone (25 mg bid) on the weekends in order to avoid the need for a diuretic.

Using multiples of 40 mg, the patient would be treated as follows: 80 mg quinapril with 0.05 mg (50 mcg) Florinef (i.e. the new combination pill), plus 40 mg quinapril without Florinef (i.e. the existing quinapril pill), b.i.d.

It may be that an even higher dose of ACE inhibitor, e.g. 2.5 mg/kg/day or even 3 or 4 mg/kg/day quinapril (or a proportional amount of ramipril, with 0.5 mg ramipril being bioequivalent to 2.0 mg quinapril) will result in even better patient outcomes. Interestingly, despite the higher dose of ACE inhibitor, the same daily dose of Florinef can result in adequate control of serum potassium (i.e. below 5.3 mEq/l). For such a high desired dose of ACE inhibitor, new dosage forms will be needed, but with the same requirement that the total daily dose of Florinef not exceed 0.1 mg/day. As an example, if the patient is to receive 4 mg/kg/day quinapril (1 mg/kg/day ramipril), he would need to take the following combination dosage forms:

Quinapril 200 mg with Florinef 0.05 mg (50 mcg) b.i.d., or

Ramipril 50 mg with Florinef 0.05 mg (50 mcg) b.i.d, to achieve both the desired ACE inhibitor and Florinef doses.

Similarly, patients with hypokalemia can have their serum potassium modified upwards by manipulation of the renin-angiotensin system. These patients currently receive potassium in the form of oral potassium chloride supplementation. Losartan, an angiotensin II receptor antagonist (ARB, for Angiotensin II Receptor Blocker), when combined with an ACE inhibitor (either hydrophilic or hydrophobic, see partial list above), results in elevation of serum potassium.

A patient with hyperaldosteronism characteristically has hypertension and hypokalemia, both the results of excess serum aldosterone concentration. The hypertension is preferably treated with an ACE inhibitor, and hypokalemia with addition of an ARB such as losartan, candesartan, valsartan, etc.

In patients with congestive heart failure (CHF), ACE inhibitors are now indicated as well as ARB's, since cardiac angiotensin II is thought to come 50% from ACE and 50% from non-specific dipeptidases such as chymase. Most patients with CHF also require diuretics to control volume overload. As a result of diuretic use, their serum potassium concentration often falls below 4.0 mEq/l, the ideal clinical value. Many times, CHF patients have cardiac arrhythmias which are exacerbated by hypokalemia, such as atrial fibrillation, supraventricular tachycardias, ventricular tachycardias, atrio-ventricular block of various degrees, and the like. Clinically, it is important to normalize their serum potassium concentration to 4 mEq/l.

Traditionally, this has been done by giving the patient oral KCl, in the form of liquid or tablets. Liquid KCl, although inexpensive, tastes terrible, creating patient non-compliance. Furthermore, there is the risk of high peak serum potassium concentration from neat KCl solution. A delayed-release pill has been developed, but it has been linked with occasional gastric distress, and even small bowel perforation, presumably because the high local concentration of K+ from a pill releasing KCl next to the gastric or small bowel mucosa causes necrosis of epithelial cells and the muscularis mucosae.

An alternative to traditional potassium supplementation for these patients is simply the addition of an ARB to the ACE inhibitor. ARB is already indicated by their CHF. For example, the addition of losartan 50 mg/day to a patient already taking an ACE inhibitor who is at the clinically desired target blood pressure of 120/80, but who is also taking Lasix (furosemide) 40 mg/d and has a serum potassium of 3.0 mEq/l, for example, will normalize the serum potassium to 4.0 mEq/l without any significant change in blood pressure. This regimen will have the added benefits of eliminating the need for KCl administration, with its resultant toxic side effects (including elevated peak K+ levels from quick release preparations, and mucosal necrosis from delayed release preparations), and further reduction in tissue angiotensin II effects by blocking angiotensin II at its receptor (with the ARB such as losartan) as well as by its production by ACE. This is important because no inhibitors yet exist to block production of angiotensin II by chymase.

D. Animal Feed or Pharmaceutical Formulations

Hydrophobic ACE inhibitors such as quinapril or ramipril can be put in the animal feed of species subject to renal failure such as cats and dogs, or other ACE-related diseases such as degenerative joint disease in dogs (ACE DD odds ratio 1.25 for black men with DJD, odds ratio 1.07 for white men). The amount of quinapril in the animal feed can be calculated, based on daily intake, to yield a final dose of 2-10 mg/kg/day. The optimal dose for delaying age-dependent diseases, and prolonging lifespan, will need to be arrived at by trial and error, which those skilled in the art can readily perform.

Other forms of ACE inhibitor for household pets or zoological animals include:

a chewable tablet;

a chewable tablet made in combination with another substance routinely given to the animal, such as a heart-worm pill for dogs, or an anti-flea compound for dogs and/or cats;

a sustained release tablet, that could be given once a week, once a month, or even less frequently.

The present invention will be further understood by reference to the following non-limiting examples.

### EXAMPLE 1

### Calculation of Benefit of Increased ACE Inhibitor Dosages

Observational studies have indicated dramatically improved patient outcomes when treating a subset of these diseases with an increased dose of a hydrophobic ACE inhibitor (ACE INHIBITOR) such as quinapril 2 mg/kg/day(*), or ramipril(“), in particular, ESRD/HTN, ERSD/NIDDM, ASPVD, and COPD.

The following are the expected difference in outcomes:

Outcomes data for patients with CRF due to hypertension, determined as Time to Dialysis, for patients with serum creatinine of at least 2 mg/dl at the first clinic visit:

| Caucasian men: | |
|---|---|
| Conventional Rx (Quinapril <40 mg/d) | 4.3 yr |
| Quinapril >80 mg/d + Florinef | 17.4 yr |

| African American men: | |
|---|---|
| Conventional Rx (Quinapril <40 mg/d) | 3.6 yr |
| Quinapril >80 mg/d + Florinef | 14.8 yr |

The following are the expected differences in outcomes for patients with CRF due to NIDDM, determined as Time to Dialysis, for patients with serum creatinine of at least 2 mg/dl at the first clinic visit:

| Caucasian men: | |
|---|---|
| Conventional Rx (Quinapril <40 mg/d) | 2.7 yr |
| Quinapril >80 mg/d + Florinef | 4.0 yr |

| African American men: | |
|---|---|
| Conventional Rx (Quinapril <40 mg/d) | 3.3 yr |
| Quinapril >80 mg/d + Florinef | 9.3 yr |

The following are the expected differences in outcomes for patients with ADPKD, determined as Time to Dialysis, for patients with serum creatinine of at least 2 mg/dl at the first clinic visit:

| Caucasian men: | |
|---|---|
| Conventional Rx (Quinapril <40 mg/d) | 8.8 yr |
| Quinapril >80 mg/d + Florinef | 8.9 yr |

These clinical data indicate that ACE is not a modifying gene for progression of ADPKD, which has also been suggested by genetic studies [van Dijk, et al., Nephrol Dial Transplant. 15(6):836-9 (2000).

### EXAMPLE 2

### Actual Outcomes for Two Patients with ASPVD Treated with High Dose ACE Inhibitor

A 74 year old white male and a 73 year old black male, both heavy smokers with HTN, severe ASPVD. They were seen because serum creatinine was approximately 3 on the day of scheduled femoral-popliteal revascularization.

They were begun on Quinapril 2 mg/kg/d in addition to vigorous blood pressure and lipid lowering; surgery canceled.

Revascularization was delayed four to five years in both cases.

### EXAMPLE 3

### Actual Outcome for Patient with COPD

A 65 year old white male with end-stage COPD, FEV1 0.87 L, on 2 L/min oxygen, 1 ppd, first seen with BP 104/60, 4+ edema despite Lasix 40 mg qd, ie severe R-sided failure.

He was begun on ramipril 2.5 mg/d 10/95 as outpatient. Two weeks later he had BP 180/110; currently on 600 mg/d ramipril with BP 135/80. Still smoking ½ to 1 package of cigarettes per day. No other changes in medications. FEV1 0.83 (8/99), 25 lb. non-fluid wt. Gain. Developed CHF with Amlodipine added for BP control; responded to increased dose of Lasix. Hospitalized for CHF but not for COPD in a period of over five years.

I claim:

1. A method for treating diseases associated with a excess of angiotensin enzyme activity comprising administering an effective dosage of an ACE inhibitor to inhibit tissue ACE.

2. The method of claim 1 wherein greater than 95% of the tissue ACE is inhibited.

3. The method of claim 1 wherein a dosage of a hydrophobic ACE inhibitor equivalent to 80 mg/day or greater quinapril is administered to a patient.

4. The method of claim 3 further comprising administering aldosterone with the ACE inhibitor.

5. The method of claim 3 wherein a hydrophobic ACE inhibitor is administered in a dosage equivalent to quinapril, from 20 mg once a day once daily, to 20 mg twice a day after one to two months, to 40 mg twice a day after an additional one to two months, to 80 mg twice a day after an additional one ot two months.

6. The method of claim 1 wherein the disease is selected from the group consisting of end-stage renal disease with hypertension, end-stage renal disease with non-insulin dependent diabetes mellitus (type II diabetes mellitus), end-stage renal disease due to focal segmental glomerulosclerosis (FSGS), membranous glomerulonephritis (GN), membranoproliferalive GN (MPGN), kidney stones, IgA GN, obstructive uropathy, and acquired renal cystic disease of end-stage renal disease.

7. The method of claim 6 to delay progression of renal failure due to hypertension or type II NIDDM, administer hydrophobic ACE inhibitor before adding any additional anti-hypertensive agent.

8. The method of claim 1 wherein the ACE inhibitor is administered in a dosage equivalent to ramipril dose of 0.5 mg/kg/day or quinapril 2 mg/kg/day.

9. The method of claim 1 wherein the disease to be treated is selected from the group consisting of cigarette abuse, asthma, pulmonary hypertension, pulmonary embolism, left ventricular hypertrophy, atherosclerotic peripheral vascular disease, deep vein thrombosis, and chronic obstructive pulmonary disease or emphysema.

10. The method of claim 1 wherein the disease to be treated is selected from the group consisting of Obesity (BMI>30), Cholesterol>200, hypertriglyceridemia, hypercholesterolemia, and mixed hyperlipidemia, NIDDM/retinopathy, and NIDDM/neuropathy.

11. The method of claim 1 wherein the disease to be treated is selected from the group consisting of scleroderma, lupus (SLE), gout, hypothyroidism, tertiary hyperparathyroidism in end-stage renal disease (ESRD), the need for frequent de-clotting of vascular access in ESRD patients, Paget's disease of bone, osteoporosis, allergy to penicillin or sulfa, allergic sinusitis or rhinitis, pelvic inflammatory disease, prevention of hip fractures, eczema, psoriasis, basal cell skin cancer, Osteoarthritis (DJD), degenerative disc disease, and Rheumatoid arthritis.

12. The method of claim 1 wherein the disease to be treated is selected from the group consisting of GERD, gallstones, peptic ulcer disease, hiatal hernia, diverticulosis, gastritis, pancreatitis, ascites, alcoholic hepatitis, cirrhosis, cholecystitis, diverticulitis, irritable bowl syndrome, inflammatory bowel disease, and inguinal hernia.

13. The method of claim 1 wherein the disease to be treated is solid tumors, leukemias, and lymphomas.

14. The method of claim 1 wherein the disease to be treated is selected from the group consisting of stroke (CVA), TIA/s/p CEA, seizures, Alzheimer's disease, dementia (non-specific), headaches, migraine headache, parkinsonism, and multi-infarct dementia.

15. The method of claim 1 wherein the disease to be treated is Bipolar affective disorder, schizophrenia, depression, anxiety, and drug abuse.

16. The method of claim 1 wherein the disease to be treated is selected from the group consisting of glaucoma and cataracts.

17. The method of claim 1 wherein the disease to be treated is presbycusis.

18. The method of claim 1 wherein the disease to be treated is viral hepatitis A, viral hepatitis B, tuberculosis, HIV infection or complications of HIV infection such as HIV-associated nephropathy and AIDS.

19. The method of claim 1 wherein the ACE inhibitor is administered in combination with a compound such as fludrocortisone acetate to a patient who has a serum K+ concentration above 4.5 mEq/l before initial dosing.

20. The method of claim 1 wherein the ACE inhibitor is administered with an angiotensin II receptor antagonist.

21. The method of claim 1 wherein the ACE inhibitor is administered with a diuretic.

22. The method of claim 1 wherein the ACE inhibitior is a hydrophilic ACE inhibitors selected from the group consisting of captopril, enalapril, and lisinopril.

23. The method of claim 1 wherein the ACE inhibitor is a hydrophobic ACE inhibitors selected from the group consisting of ramipril, benazepril, and quinapril.

24. The method of claim 1 comprising treating a non-human animal.

25. A method of determining if a disease can be treated with ACE inhibitors comprising calculating the odds ratio of association between a disease and the ACE D/D genotype and determining if the odds ratio is greater than 1.0

26. The method of claim 25 wherein the odds ratio is 2.0 or greater.

27. The method of claim 25 wherein the odds ratio is between 1.0 and less than 2.0.

28. A dosage formulation for treating disorders associated with the ACE D/D genotype comprising an amount of an ACE inhibitor effective to inhibit greater than 95% tissue ACE or a dosage delivering greater than 80 mg/day of an ACE inhibitor such as quinapril.

29. The dosage formulation of claim 28 in the form of tablets providing a dosage selected from the group consisting of 80, 100 and 200 mg quinapril.

30. The dosage formulation of claim 28 equivalent to a ramipril dose of 0.5 mg/kg/day or quinapril 2 mg/kg/day.

31. The dosage formulation of claim 28 in a sustained or controlled release carrier.

32. The dosage formulation of claim 31 comprising a dosage equivalent to 200 mg quinapril in a carrier providing sustained release over a period of up to one day.

33. The dosage formulation of claim 31 comprising a dosage equivalent to 100 mg SR quinapril in a carrier providing sustained release over a period of up to one day.

34. The dosage formulation of claim 28 in the form of tablets providing a dosage selected from the group consisting of 20 mg, 50 mg, 100 mg and 200 mg ramipril.

35. The dosage formulation of claim 28 comprising an ACE inhibitor in an amount effective to inhibit tissue ACE and a diuretic.

36. The dosage formulation of claim 28 comprising an ACE inhibitor in combination with an angiotensin receptor blocker.

37. The dosage formulation of claim 28 comprising an ACE inhibitor in combination with a compound increasing aldosterone levels or the effects thereof.

38. The dosage formulation of claim 37 comprising Quinapril 40 mg with 0.05 mg Florinef, or Quinapril 80 mg with 0.05 mg Florinef.

39. A formulation of an ACE inhibitor for administration to an animal comprising a carrier selected from the group consisting of animal feed and chewable tablets.
